(19) United States
(12) Patent Application Publication
(10) Pub. No.: US 2025/0025357 A1
(43) Pub. Date: Jan. 23, 2025

(54) WEARABLE ABSORBENT ARTICLE

(71) Applicant: Coterie Baby, Inc., New York, NY (US)

(72) Inventors: Juan Barth, Sabaneta (CO); Carlos E. Richer, Conroe, TX (US); Natalia Richer, Conroe, TX (US)

(21) Appl. No.: 18/778,448

(22) Filed: Jul. 19, 2024

Related U.S. Application Data
(60) Provisional application No. 63/514,719, filed on Jul. 20, 2023.

(51) Int. Cl. A61F 13/62; A61F 13/49; A61F 13/56

(52) U.S. Cl. CPC A61F 13/627; A61F 13/49015; A61F 13/5638

(57) ABSTRACT

A wearable absorbent article including a first fastener disposed on a first elastic side panel and a second fastener disposed on a second elastic side panel. Each of the first and second fasteners includes a fastening component having at least one first strip of fastening material and a tab portion having a first portion and a second portion. The first portion of the tab portion is disposed adjacent to the fastening component and has at least one second strip of fastening material. The second portion of the tab portion extends from the first portion and beyond a lateral end of the respective one of the first and second elastic side panels, and has at least one third strip of fastening material. The second portion has a length that is less than a length of the first portion.

### RELATED APPLICATION

This application is a non-provisional claiming priority to and the benefit of U.S. Provisional Patent Application No. 63/514,719, filed Jul. 20, 2023 and entitled Wearable Absorbent Article, the contents of which are incorporated herein by reference in their entirety.

### FIELD OF THE INVENTION

The present invention generally relates to wearable absorbent articles, such as disposable diapers and underpants-type articles, and more particularly, wearable absorbent articles with an adjustable fastening means to accommodate application, wear and removal of the absorbent article on a user.

### BACKGROUND OF THE INVENTION

Disposable diapers, pull-on diapers, training pants, adult diapers and disposable absorbent underpants-type articles are known in the art and generally include an absorbent core for retaining excreted bodily fluids. Such articles also generally include an elasticized waist band to provide comfort to the wearer, as well as adhesive tabs for facilitating application of the diaper to the intended wearer—typically an infant or toddler who generally cannot apply the absorbent article to themselves. Such adhesive tabs are useful to a parent to be able to apply the diaper to an infant or toddler who may be difficult to keep still while putting on the diaper.

Conventional adhesive tabs are further designed to ensure a tight fit to the child so that excreted bodily fluids do not leak from the leg holes or waist of the diaper. In practice, the adhesive tabs for such diapers are generally for one-use, meaning that once adhered and fastened, it is difficult to adjust the fit of the diaper because detaching the adhesive tabs reduces the adhesiveness of the tabs, risks damage to the diaper, and may generally affect the intended sealed fit of the diaper to the infant or toddler. Thus, adhesive tab designs for conventional diapers are more focused on ensuring a secure attachment than on ensuring a comfortable fit or a reusable option wherein the absorbent article can be removed by undoing the tabs and then reapplied without risk of the means of attachment being denigrated in any way.

By comparison, pull-up diapers, which are generally designed for older children, typically only include an elasticized waist because the child can “pull-up” the article themselves and therefore do not need the side tabs. Additionally, when the child is done wearing the pull-up diaper—which is generally designed for a single use—the child can easily pull down the diaper or simply tear it off for disposal. Adult diapers are similarly designed with an elasticized waist to mimic undergarments and are likewise applied in a similar manner. An issue with these designs is that comfortable fit is often compromised as such articles are typically only provided in set sizes, with the elastic waistband provided to accommodate users of varying sizes. However, if the size of the article is too small, the fit can be tight and uncomfortable, and the elastic waistband can press on the wearer's waist and abdomen and leave indentations or marks.

Some conventional pull-up diapers are provided with refastening systems to allow for adjustment and proper positioning of the diaper on the wearer. Conventional refastening systems include a wide rectangular strip of hooks attached to the stretchable edges of the pant's waistband. These edges located at the ends of the waistband are made of light nonwoven material and elastic threads of synthetic rubber, enabling the waistband to stretch and fit the baby's waist circumference. When the pant is worn, it initially conforms to the pre-fitted waist size provided by the manufacturer as a standard size. The refastening system allows for further adjustments to ensure the waist circumference fits the baby perfectly, as described above.

There are a number of known issues with such pant-like wearable absorbent products with refastening systems. For example, the hook strip is attached to the flexible edges of the waistband, and thus grasping these edges to open the pant and reposition it is difficult and inaccurate because the edges elongate when force is applied, making precise adjustment challenging. Further, the repositioning of the hook is often inaccurate, as it may not align properly with the manufacturer's intended perpendicular position. This requires multiple attempts to refasten the system correctly, which is further complicated by the baby's movements. Still further, nighttime changes are even more challenging due to dim lighting conditions.

Another significant issue with conventional re-fastenable pant-like diapers relates to comfort of the wearer. Specifically, the positioning of the mechanical hook at the end of the elasticized waistband has been found to be unsafe for some babies. The rectangular hook strip lies over the top hinge of the wearer's hip (i.e., the “hip crease”), and because of the sharp edges and pointed corners of the hook strip, this can pose a hazard to the wearer's skin in the form of irritation or injuries.

In view of the foregoing, there is a need for a wearable absorbent article design having an adjustable fastening system to accommodate application, wear and removal of the absorbent article on a user, including infants, toddlers, children and adults, whereby the fastening system can be attached, detached and adjusted for multiple uses as desired without compromising fit and comfort of the wearable absorbent article. Further, there is a need for such an adjustable fastening system that will not damage the body of the wearable article when applied and detached. Still further, there is a need for such an adjustable fastening system which will ensure a tight seal when applied to the wearer and attached to fit the wearer's body, and thus will not be too tight and restrictive when the wearer is moving but will not risk unintended detachment during wear and use.

### SUMMARY OF THE INVENTION

An object of the present invention is to provide a wearable absorbent article with an adjustable fastening system that overcomes the problems and drawbacks associated with conventional wearable absorbent articles, such as conventional disposable diapers, pull-up diapers, adult diapers and underpants-type articles, and therefore significantly improve the utility of such wearable absorbent articles.

Another object of the present invention is to provide a fastening system for an wearable absorbent article that minimizes discomfort to the wearer, and specifically by allowing the fastening system to be reduced in size compared to conventional fastening systems so that components of the fastening system do not contact parts of the wearer's body during use that might otherwise cause injury, such as, for example, chafing or other types of irritation.

Another object of the present invention is to provide a fastening system that maintains acceptable levels of shear and peel strength even while reducing or eliminating injury to the wearer.

A wearable absorbent article according to an exemplary embodiment of the present invention comprises: a chassis comprising: a backsheet; a topsheet; and an absorbent core disposed between the backsheet and the topsheet; a first elastic side panel extending from a first side of the chassis; a second elastic side panel extending from a second side of the chassis; a front waist portion defined by a first longitudinal end portion of the chassis; a back waist portion defined by a second longitudinal end portion of the chassis and at least a portion of the first elastic panel and at least a portion of the second elastic panel; a first fastener disposed on the first elastic side panel; and a second fastener disposed on the second elastic side panel, each of the first and second fasteners comprising: a fastening component attached to a first lateral end portion of the respective one of the first and second elastic side panels, the fastening component comprising at least one first strip of fastening material; and a tab portion attached to a second lateral end portion of the respective one of the first and second elastic side panels, the second lateral end portion being disposed further from a longitudinal axis of the chassis as compared to the first lateral end portion, the tab portion comprising: a first portion disposed adjacent to the fastening component, the first portion comprising at least one second strip of fastening material; and a second portion extending from the first portion and beyond a lateral end of the respective one of the first and second elastic side panels, the second portion comprising at least one third strip of fastening material, the second portion having a length that is less than a length of the first portion, the fastening components being configured to attach to the front waist portion to place the wearable absorbent article in a closed configuration.

In an exemplary embodiment, the fastening material are hooks of a hook-and-loop fastening system.

In an exemplary embodiment, each tab portion has a total hook area defined by a sum of all hook areas in both the first and second portions of the tab portion, and the hook area in the second portion makes up at least 50% of the total hook area.

In an exemplary embodiment, the hook area in the second portion makes up at the most 75% of the total hook area.

In an exemplary embodiment, the fastening material is adhesive.

In an exemplary embodiment, the at least one second strip of fastening material has a width that is larger than a width of the at least one third strip of fastening material.

In an exemplary embodiment, a length of the at least one first strip of fastening material is equal to a length of the at least one second strip of fastening material.

In an exemplary embodiment, a length of the at least one first strip of fastening material is greater than a length of the at least one third strip of fastening material.

In an exemplary embodiment, the at least one third strip of fastening material comprises at least two third strips of fastening material.

In an exemplary embodiment, the wearable absorbent article is a pull-up diaper.

In an exemplary embodiment, the first portion of the tab portion comprises a backing, and a thickness of the backing is up to 75% of a height of the hooks.

In an exemplary embodiment, each of the first and second fasteners has a shear strength of 8.50 kg-force to 11.00 kg-force.

In an exemplary embodiment, each of the first and second fasteners has a peel strength of 1.70 kg-force to 2.20 kg-force.

### BRIEF DESCRIPTION OF THE DRAWINGS

FIG. 1 is a planar view of a wearable absorbent article in the open configuration according to an exemplary embodiment of the present invention;

FIG. 2 is a cross-section of a wearable absorbent article taken along the line A-A in FIG. 1 according to an exemplary embodiment of the present invention;

FIG. 3 is a perspective view of a wearable absorbent article in an open configuration according to an exemplary embodiment of the present invention;

FIG. 4 is a planar view of a tab portion of a fastener according to an exemplary embodiment of the present invention;

FIG. 5 is a perspective view showing a user grabbing a tab portion of a fastener according to an exemplary embodiment of the present invention;

FIG. 6 is a perspective view of a wearable absorbent article in a closed configuration according to an exemplary embodiment of the present invention;

FIG. 7 is a chart showing peel force required to separate fastening systems in accordance with Comparative Examples;

FIG. 8 is a chart showing shear force required to separate fastening systems in accordance with Comparative Examples;

FIG. 9 is a chart showing peel force required to separate fastening systems in according with Comparative Examples and also in accordance with an Example of the present invention; and

FIG. 10 is a chart showing shear force required to separate fastening systems in according with Comparative Examples and also in accordance with an Example of the present invention.

### DETAILED DESCRIPTION

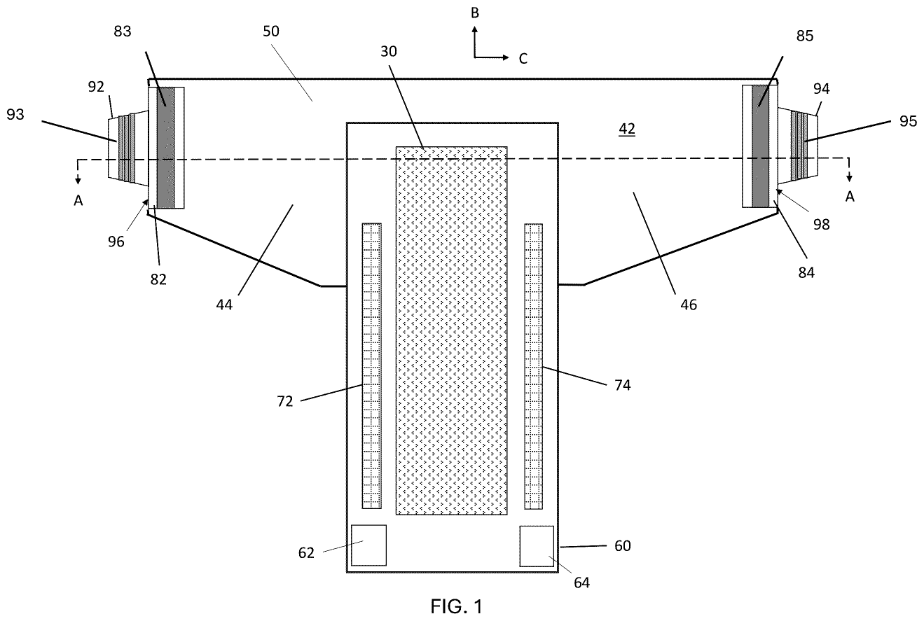

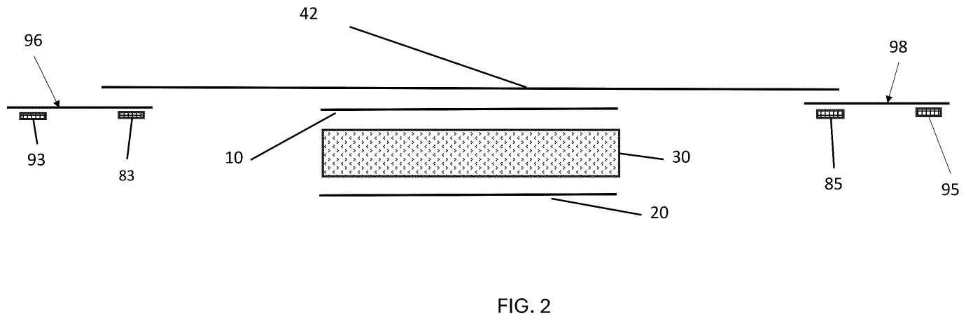

FIGS. 1 and 2 shows a plan view and a cross-sectional view, respectively, of a pull-up diaper, generally designed by reference number 1, according to an exemplary embodiment of the present invention. The diaper 1 extends generally in a longitudinal or machine-direction B and lateral or cross-direction C. The diaper 1 is made up of a backsheet 10, a topsheet 20, an absorbent core or insert 30, and an elastic portion 42 made up of two elastic side panels 44, 46. The absorbent core 30 is disposed between the backsheet 10 and topsheet 20. The topsheet 20 is positioned closest to the skin when placed on the wearer, while the backsheet 10 is positioned furthest from the skin.

The absorbent core or insert 30 may be formed or cut out from rolls of absorbent materials. As used herein, the term “absorbent core or insert” refers to a material or combination of materials suitable for absorbing, distributing, and storing aqueous fluids such as urine, blood, menses, and water found in body exudates. The size and shape of the absorbent core can be altered to meet absorbent capacity requirements; and to provide comfort to the wearer/user. The absorbent core suitable for use in the present invention can be any liquid absorbent material known in the art for use in absorbent articles, provided that the liquid absorbent material can be configured or constructed to meet absorbent capacity requirements. Nonlimiting examples of liquid absorbent materials suitable for use as the absorbent core include comminuted wood pulp, which is generally referred to as airfelt; creped cellulose wadding; absorbent gelling materials including superabsorbent polymers, such as hydrogel-forming polymeric e g agents; chemically, stiffened, modified, or cross-linked cellulose fibers; meltblown polymers including coform; synthetic fibers including crimped polyester fibers; tissue including tissue wraps and tissue laminates; capillary channel fibers; absorbent foams; absorbent sponges; synthetic staple fibers; peat moss; or any equivalent material; or combinations thereof, as is well known in the art of making absorbent products such as sanitary napkins, pantiliners, incontinence pads; and the like.

The core or insert 30 is placed on a top surface of a backsheet 10. Backsheets are materials that generally are liquid impermeable, but may be moisture vapor permeable (breathable). Backsheets are used in absorbent products on a surface of the product that is distal to the user's body. The backsheet can be any known or other effective backsheet material, provided that the backsheet prevents external leakage of exudates absorbed and contained in the protective underwear. Flexible materials suitable for use as the backsheet include, but are not limited to, woven and nonwoven materials, laminated tissue, polymeric films such as thermoplastic films of polyethylene and/or polypropylene, microporous films, composite materials such as a film-coated nonwoven material, or combinations thereof, as is well known in the art of making absorbent products, such as sanitary napkins, pantiliners, incontinence pads; and the like.

The absorbent core or insert 30 is typically attached to the backsheet 10 with an adhesive. Suitable adhesives are known in the art and include hot melt adhesives, emulsion polymer adhesives and the like.

The topsheet 20 or cover is placed on top of the core or insert 30 and attached to the core or insert 30 and backsheet 10 with adhesive, ultrasonic bonding or combinations thereof, forming a chassis. Suitable topsheets are compliant, soft feeling, and non-irritating to the body of the wearer. Suitable topsheet materials include a liquid pervious material that is oriented towards and contacts the body of the wearer, thereby permitting body discharges to rapidly penetrate through the topsheet without allowing fluid to flow back through the topsheet to the skin of the wearer. A suitable topsheet can be made of various materials, such as woven and nonwoven materials; apertured film a materials including apertured formed thermoplastic films, apertured plastic films, and fiber-entangled apertured films; hydro-formed thermoplastic films; porous foams; reticulated foams; reticulated thermoplastic films; thermoplastic scrims; or combinations thereof, as is well known in the art of making absorbent products such as sanitary napkins, pantiliners, incontinence pads, protective and the like.

The chassis may include additional materials such as acquisition distribution layers, transfer layers, secondary absorbent layers and the like.

The elastic portion 42 including the elastic side panels 44, 46 are attached to the chassis. Any elastic material known in the art of absorbent articles may be useful to form the elastic portion. Suitable elastic materials include laminates of elastic films with nonwovens, laminates of elastic strands with nonwovens and the like. The elastic portion 42 may be attached to the chassis by adhesive, ultrasonic bonding or a combination thereof. The length, width and shape of the side panels may be designed to make training pants of different sizes. The training pants have a more underwear like appearance.

The diaper 1 includes a back waist portion 50 and a front waist portion 60. In exemplary embodiments, the back waist portion 50 extends further in the lateral direction C as compared to the front waist portion 60. The back waist portion 50 is made up of the elastic portion 42 including the elastic side panels 44, 46. The elastic side panels 44, 46 allow the back waist portion 50 to be stretched for better fit of the diaper 1 on the wearer. In exemplary embodiments, the elastic side panels may together form a unitary structure or may be separate from one another. The diaper 1 also includes a pair of elasticated leg openings, i.e., a right elasticated leg opening 72 and a left elasticated leg opening 74, that fit snugly against the crotch region of the wearer in order to prevent leakage of urine or other body exudates through the leg openings. In exemplary embodiments, the front waist portion 60 is made up of the backsheet 10, which may be a laminate of a polyethylene film and nonwoven material.

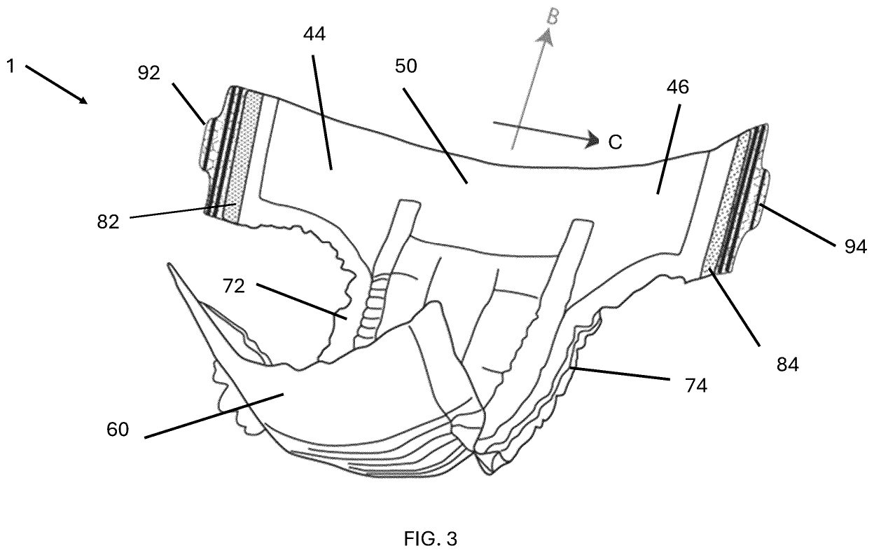

The diaper 1 further includes a first fastening component 82 and a second fastening component 84, each attached to a lateral end portion of a respective one of the elastic side panels 44, 46. The fastening components 82, 84 may include hook elements as part of a hook-and-loop fastening system. The hook elements may be arranged in an area having a shape of a rectangular strip 83, 85 extending primarily in the machine direction B. The first and second fastening components 82, 84 may have multiple hooks strips. The rectangular strips 83, 85 may have a width of 2 mm to 20 mm and a length of 20 mm to 150 mm and, in a specific exemplary embodiment, the rectangular strip has a width of 15 mm and a length of 120 mm. Corresponding areas 62, 64 with loop elements may be disposed at the front waist portion 60 to allow the front and back waist portions 50, 60 to be attached to one another so that the diaper 1 can be re-configured from an open configuration (FIG. 3) to a closed or worn configuration (FIG. 6). Alternatively, the front waist portion 60 may be devoid of corresponding loop elements, in which case the fastening components 82, 84 may be attached directly to the front waist portion 60.

In exemplary embodiments, the diaper 1 further includes a first tab portion 92 and a second tab portion 94, each disposed adjacent to a respective one of the fastening components 82, 84 at a more lateral position as compared to the position of the fastening component 82, 84. The tab portions 92, 94 may form unitary structures with the fastening components 82, 84, or may be separate elements. The tab portions 92, 94 are also provided with hook elements for attachment to the front waist portion 60. The hook elements may be arranged in an area having a shape of a rectangular strip 93, 95 that extends primarily in the machine direction B. The tab portions 92, 94 may have multiple hook strips, with at least one hook strip having a length that is less than the length of the hook strips of the first and second fastening components. In an exemplary embodiment, the length of at least one hook strip of the tab portions 92, 94 is half the length of the hook strips of the fastening components 82, 84. As shown most clearly in FIG. 6, the tab portions 92, 94 extend beyond the distal ends of the back waist portion 50. The first fastening component 82 and the first tab portion 92 together form a first fastener 96 and the second fastening component 84 and the second tab portion 94 together form a second fastener 98.

In an exemplary embodiment, the first and second fastening components 96, 98 have a peak peel strength of 1.70 kg-force to 2.20 kg-force and a peak shear strength of 8.50 kg-force to 11.00 kg-force.

The tab portions 92, 94 provide the diaper 1 with a number of advantages compared to conventional pull-up or training pant-type diapers. The flexibility of the elastic side panels 44, 46 permits the tabs portions 92, 94 to be brought around the wearer's waist, back-to-front, for engagement with the front section of the article. The addition and design of the tab portions 92, 94 provide stiffness to support the shape of the back waist portion 50, which is stretched when donning the wearable article and facilitates mechanical fastening of the tab portions 92, 94 to the front waist section 60. The tab portions 92, 94 also provide a clear and simplified location to grasp the elastic side panels 44, 46, either to fasten or unfasten the wearable article to the user. Still further, the tab portions 92, 94 provide for quick adjustment of the fit of the wearable article without needing to completely remove the article or replace the article after the fastening components 82, 84 had already engaged to the front waist portion 60. Indeed, with the tab portions 92, 94 in place secured to the front waist portion 60, they can be disengaged one at a time to reposition without risking damage to the front waist portion 60 or affecting the adhesiveness of the tab portions 92, 94 to the chassis.

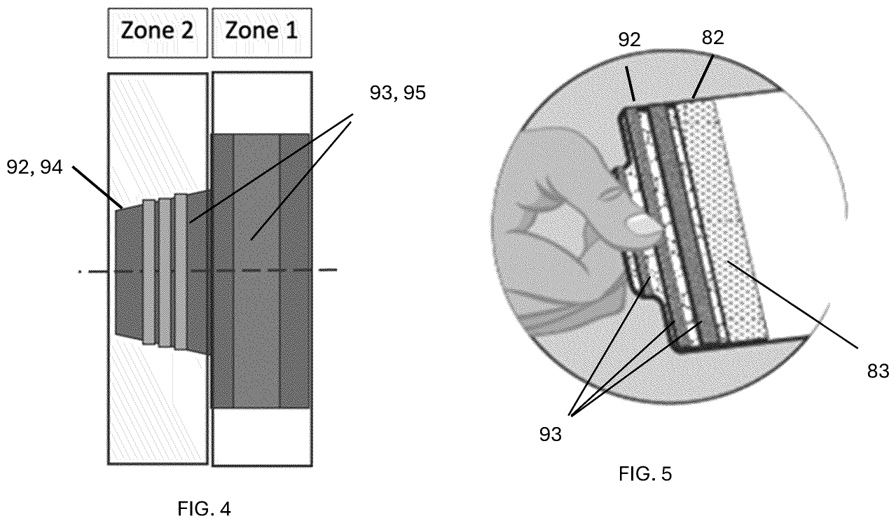

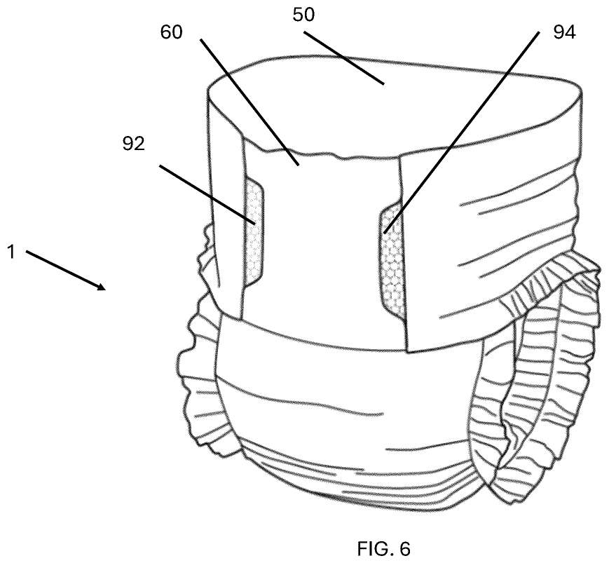

As shown in FIGS. 4, in exemplary embodiments, each of the tab portions 92, 94 is provided with a first zone and a second zone disposed further in the cross-direction C as compared to the first zone.

The first zones provide rectangular areas that extend mainly in the machine-direction B. The first zones are intended to provide a “soft” means of attachment to the front waist portion 60. In exemplary embodiments, the first zones are provided with mushroom type hooks which are primarily responsible for providing the fastening arrangement with enhanced shear strength. In exemplary embodiments, each of the first zones includes at least one strip of hooks, and in the case of multiple strips, the strips are spaced from one another in the cross-direction C.

The second zones extend outwards from the distal ends of the elastic side panels 42, 44 and include regions of the tab portions 92, 94 that extend primarily in the cross-direction C. The main attribute of the second zones is to reinforce the peel strength of the fastening arrangement. The second zones likewise include adhesive strips—again preferably of the hook-and-loop style—to allow for easy attachment and detachment of the tab portions 92, 94 to and from the front waist portion 60. In exemplary embodiments, each of the second zones includes at least one strip of hooks, and in preferred exemplary embodiments include at least two strips of hooks. In the case of multiple strips, the strips are spaced from one another in the cross-direction C. The strips of hooks in the second zones are preferably smaller in width as compared to the strips of hooks in the first zones.

In exemplary embodiments, the second zone of each tab portion 92, 94 includes hook strips that form a hook area that is up to at least 50%, preferably up to 60%, and even more preferably up to 75% of the total hook area of the tab portion 92, 94, where the total hook area is the sum of all hook areas in both the first and second zones.

In use, the second zones of the tab portions 92, 94 can be grasped and pulled to stretch the elastic side panels 44, 46 to a desired length around the wearer. The first zones can then be attached to the front waist portion 60, and when the user is satisfied with position and placement of the first zones, the second zones can then be attached to the front waist portion 60 to reinforce the connection of the first and second fasteners 96, 98.

In accordance with exemplary embodiments of the present invention, the fasteners 96, 98 can be disengaged from the front waist portion 60 for adjustment of the fit of the diaper 1 without risking damage to the diaper 1. Further, the tab portions 92, 94 can be connected to the front waist portion 60 at any location to ensure a comfortable, but sealed, fit. In this regard, the elastic side panels 44, 46 can be moved further toward the front of the diaper 1 or positioned along the side waist area. Additionally, the elastic side panels 44, 46 can be flexed along the front waist portion 60 upward toward the waist or downward towards the crotch and the tab portions 92, 94 can then be used to secure the elastic side panels 44, 46 in place where positioned.

Additionally, the diaper 1 may be reused after removal, as desired. In this regard, the diaper 1, including the absorbent core 30, backsheet 10 and topsheet 20, may be made from materials that can be washed and dried, without damage to the adjustable fastening means or the absorbent properties of the article, and then reused.

As noted, the fastening system of the present invention provides extra rigidity and stiffness to support the shape of the back waist portion 50 when the wearable article is applied to the user. Such reinforcement reduces buckling of the elastic side panels 44, 46 during application and wear, which in turn maintains the desired seal of the wearable article. Indeed, the double zone design of the tab portions 92, 94 in accordance with the present invention ensures good reinforcement that is more substantial than just having a first zone. The second zone provides added stiffness and reinforcement to the back waist portion 50 when stretched around the user.

The design of the tab portions 92, 94 in accordance with the present invention also increases the adhesive area by increasing the backing thickness and adding more basis weight for reinforcement. The bonding of the hook-and-loop sections to the wings/extensions utilizes desirable bonding areas to increase stiffness and reinforcement without affecting the stretchability of the material or give of the wearable article during wear and use. The backing of the tab portions 92, 94 (preferably made of nonwoven material) provides additional basis weight that supports structure reinforcement and stability when the tab portions 92, 94 are stretched to position and attached for wear and use. In an exemplary embodiment, in order to provide stiffening and reinforcement, the thickness of the backing in the first zone is at least 50% of the hook height, and in a preferred exemplary embodiment, is up to 75% of the hook height. In exemplary embodiments, the fastening system provides at least 45±5 gsm nonwoven material to supplement and allow the desired structural stiffness reinforcement.

The design of the tab portions 92, 94 in accordance with the present invention also facilitates the case of opening the adjustable waist of the wearable article—for example, to remove the article or to adjust the fit. In this regard, the second zones of the tab portions 92, 94 are provided with a finger tab sections that facilitate grasping the tab portions 92, 94, stretching of the elastic side panels 44, 46 to appropriate positions, and attachment of the tab portions 92, 94 to the front waist portion 60 in a desired location and position. In this regard, the finger tabs allow for a pincer grasp of the tab portions 92, 94—i.e., using two fingers—as opposed to requiring two hands to pull and position each side of the wearable article, which is undesirable. In exemplary embodiments, the finger grip portions may be formed by a nest cut of the tab portion material.

In exemplary embodiments, the addition of the tab portions 92, 94 provides improved comfort to the wearer. Specifically, as discussed, the tab portions 92, 94 provide the fastening system with enhanced shear strength, and thus the fastening components 82, 84 can be reduced in length in the machine-direction B without reducing the shear strength to unacceptable levels at which the strength and stability of the fastening arrangement becomes compromised. For example, the bottom edges of the fastening components 82, 84 can be disposed higher up in the machine-direction B, further away from the hip crease of the wearer, thereby avoiding chafing or other skin irritation that might otherwise occur with conventional fastening systems.

The below Comparative Examples and Examples exhibit the advantages and improvements provided by exemplary embodiments of the present invention in terms of peel strength and shear strength of the fastening system. The following test methods were used:

### Test Method—Shear and Peel Stength

The peak peel force and peak shear force were tested using the ASTM D5170-98 standard test method, adapted to test the finished product instead of 1 inch cut out of raw material, and with the following additional modification: a 5 lb roller was rolled 6 times (instead of 11 lb roller, 5 times). The test measured the peak peel force and the peak shear force required to separate two portions of a sample attached by a hook and loop attachment. The test was performed at 90 degrees relative to the face of the sample to obtain peel strength and at 180 degrees relative to the face of the sample to obtain the shear strength.

### Comparative Example 1

A commercially available diaper was provided (Coterie The Pant, Size 5). The hook strips were removed from the back waist portion of the diaper. The diaper was deconstructed to obtain a portion of the back waist and a portion of the front waist of the diaper that were originally intended to be attached using the removed hook strips. For each back waist portion, a replacement hook strip was attached at the location where the original hook strip was located. The length of the replacement hook strip was 120 mm. Each back waist portion was disposed adjacent to a corresponding front waist portion and then the back waist portion was attached to the front waist portion using the replacement hook strip to form a test sample. The replacement hook strip was placed on the front waist portion initially without applying pressure and then a 5-pound roller was passed over the replacement hook strip five (5) times. Five (5) such samples were prepared and tested. The average peak peel force was 1.9 kg-force and the average peak shear force was 9.5 kg-force.

### Comparative Example 2

Five (5) samples were prepared as described in Comparative Example 1, but the lengths of the replacement hook strips in the machine direction were 100 mm. The average peak peel force was 1.8 kg-force and the average peak shear force was 9.0 kg-force.

### Comparative Example 3

Five (5) samples were prepared as described in Comparative Example 1, but the lengths of the replacement hook strips in the machine direction were 84 mm. The average peak peel force was 1.6 kg-force and the average peak shear force was 8.8 kg-force.

### Comparative Example 4

Five (5) samples were prepared as described in Comparative Example 1, but the lengths of the replacement hook strips in the machine direction were 50 mm. The average peak peel force was 1.2 kg-force and the average peak shear force was 8.0 kg-force.

### Example 1

Five (5) samples were prepared as described in Comparative Example 3 (i.e., the lengths of the replacement hook strips in the machine direction were 84 mm), but with a piece of nonwoven with an additional hook strip added to each back waist portion. The additional hook strip had a length in the machine direction of 70 mm. The additional hook strip extended from the lateral end of the back waist portion to create a finger tab type structure. The attachment between the front and back waist portions occurred at two points—a first point where the replacement hook strip attached to the front waist portion and a second point where the additional hook strip of the finger tab structure attached to the front waist portion. The average peak peel force was 2.1 kg-force and the average peak shear force was greater than 10 kg-force.

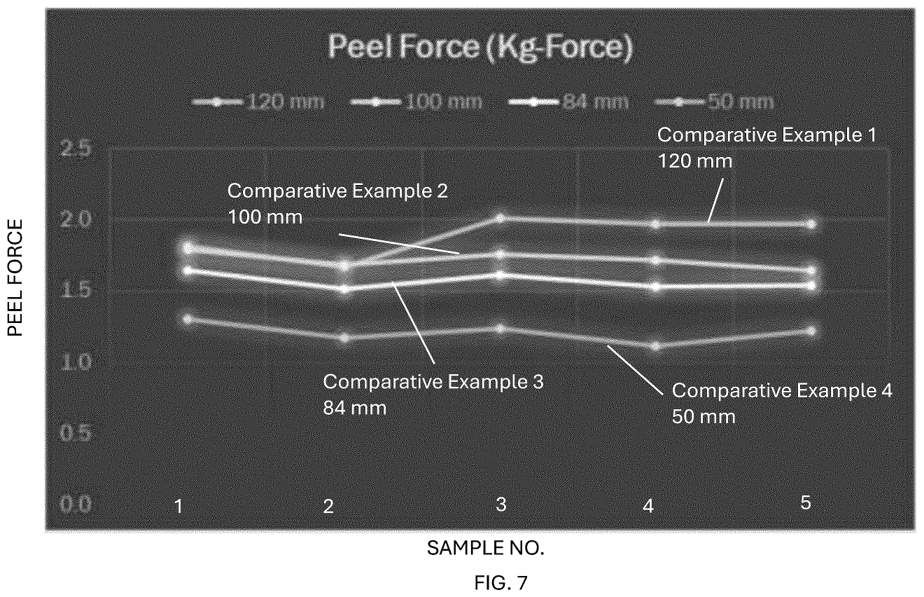

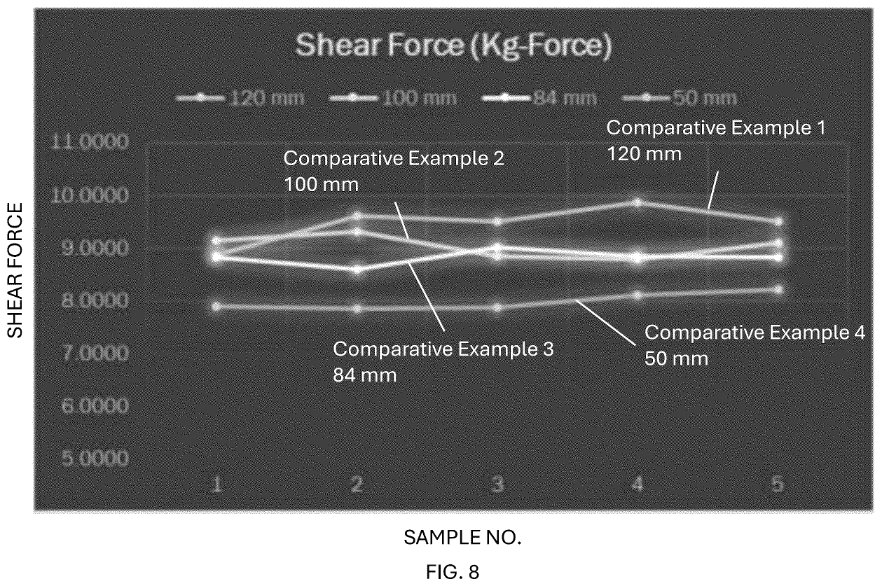

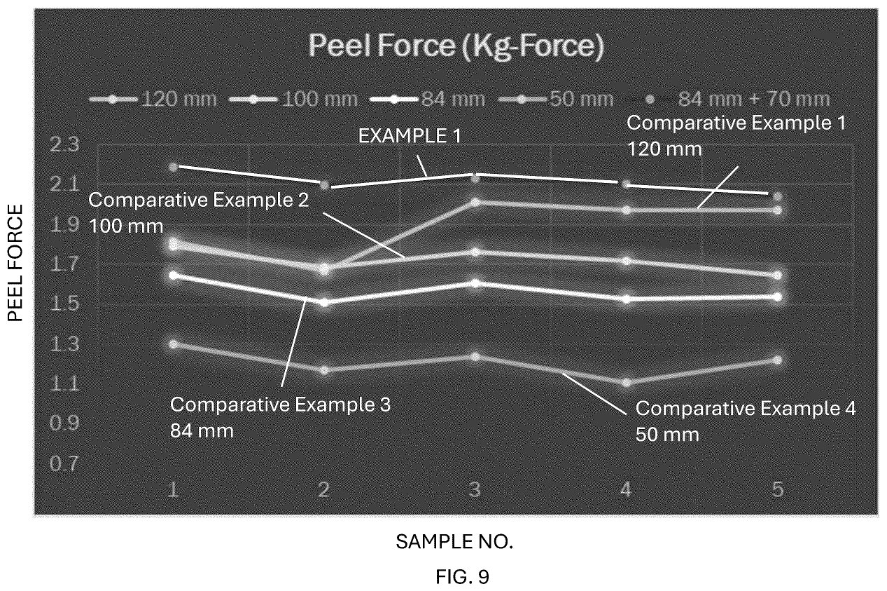

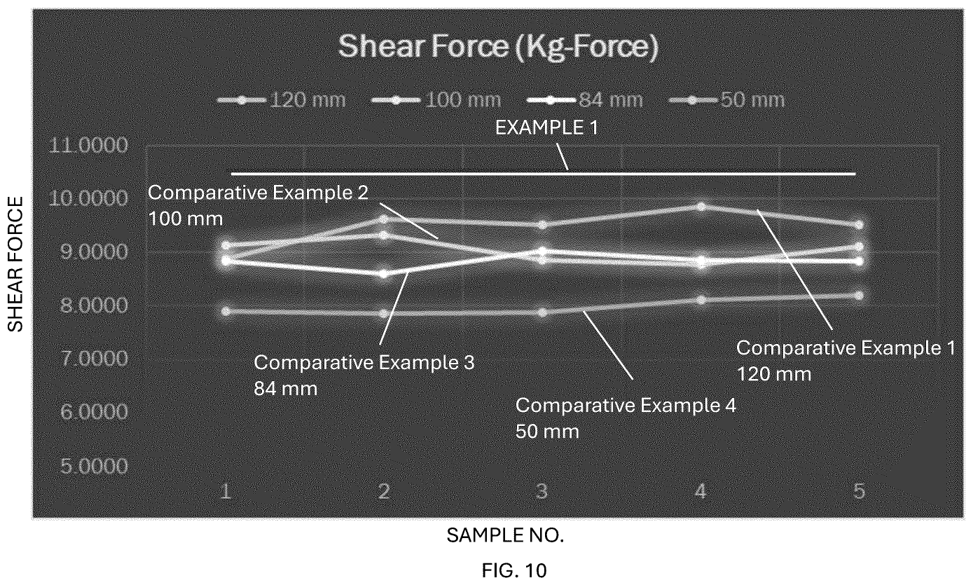

The results of Comparative Examples 1-4 and Example 1 are summarized in the charts shown in FIGS. 7-10.

The Comparative Examples demonstrate that the length of the replacement hook strip was directly related to the peel and shear strengths of the fastener attachment, as expected. However, Example 1 showed that the addition of the finger tab structure resulted in an increase in peak peel force and peak shear force as compared to all of the Comparative Examples, even in comparison to Comparative Examples 1 and 2 which had longer replacement hook strips. This showed that the addition of the finger tab type structure with the additional hook strip compensated for the relatively short replacement hook strip in terms of shear and peel strength. Accordingly, relatively short hooks strips can be used on diapers to achieve improved comfort as described in exemplary embodiments of the present invention, with the finger tabs with additional hook strips being provided to compensate for the reduction in peel and shear strengths exhibited by the shorter hook strip.

The foregoing description and accompanying images of embodiments of the present invention has been presented for the purpose of illustration and description. They are not intended to be exhaustive or to limit the invention to the specific form and design disclosed. Obvious modifications and variations are possible in light of the above disclosure. The embodiments described were chosen to best illustrate the principles of the invention and practical application thereof to enable one of ordinary skill in the art to utilize the invention in various embodiments and with various modifications as suited to the particular uses contemplated.

What is claimed is:

1. A wearable absorbent article comprising:
a chassis comprising:
a backsheet;
a topsheet; and
an absorbent core disposed between the backsheet and the topsheet;
a first elastic side panel extending from a first side of the chassis;
a second elastic side panel extending from a second side of the chassis;
a front waist portion defined by a first longitudinal end portion of the chassis;
a back waist portion defined by a second longitudinal end portion of the chassis and at least a portion of the first elastic panel and at least a portion of the second elastic panel;
a first fastener disposed on the first elastic side panel; and
a second fastener disposed on the second elastic side panel,
each of the first and second fasteners comprising:
a fastening component attached to a first lateral end portion of the respective one of the first and second elastic side panels, the fastening component comprising at least one first strip of fastening material; and
a tab portion attached to a second lateral end portion of the respective one of the first and second elastic side panels, the second lateral end portion being disposed further from a longitudinal axis of the chassis as compared to the first lateral end portion, the tab portion comprising:
a first portion disposed adjacent to the fastening component, the first portion comprising at least one second strip of fastening material; and
a second portion extending from the first portion and beyond a lateral end of the respective one of the first and second elastic side panels, the second portion comprising at least one third strip of fastening material, the second portion having a length that is less than a length of the first portion,
the fastening components being configured to attach to the front waist portion to place the wearable absorbent article in a closed configuration.

2. The wearable absorbent article of claim 1, wherein the fastening material are hooks of a hook-and-loop fastening system.

3. The wearable absorbent article of claim 2, wherein each tab portion has a total hook area defined by a sum of all hook areas in both the first and second portions of the tab portion, and the hook area in the second portion makes up at least 50% of the total hook area.

4. The wearable absorbent article of claim 3, wherein the hook area in the second portion makes up at the most 75% of the total hook area.

5. The wearable absorbent article of claim 1, wherein the fastening material is adhesive.

6. The wearable absorbent article of claim 1, wherein the at least one second strip of fastening material has a width that is larger than a width of the at least one third strip of fastening material.

7. The wearable absorbent article of claim 1, wherein a length of the at least one first strip of fastening material is equal to a length of the at least one second strip of fastening material.

8. The wearable absorbent article of claim 1, wherein a length of the at least one first strip of fastening material is greater than a length of the at least one third strip of fastening material.

9. The wearable absorbent article of claim 1, wherein the at least one third strip of fastening material comprises at least two third strips of fastening material.

10. The wearable absorbent article of claim 1, wherein the wearable absorbent article is a pull-up diaper.

11. The wearable absorbent article of claim 2, wherein the first portion of the tab portion comprises a backing, and a thickness of the backing is up to 75% of a height of the hooks.

12. The wearable absorbent article of claim 1, wherein each of the first and second fasteners has a shear strength of 8.50 kg-force to 11.00 kg-force.

13. The wearable absorbent article of claim 1, wherein each of the first and second fasteners has a peel strength of 1.70 kg-force to 2.20 kg-force.
